(19) United States
(12) Patent Application Publication
(10) Pub. No.: US 2004/0028642 A1
(43) Pub. Date: Feb. 12, 2004

(54) Cosmetic composition comprising an extract of emblica officinalis and methods of using same

(76) Inventors: Isabelle Hansenne, Westfield, NJ (US); Angelike Galdi, Westfield, NJ (US); Hani Fares, Somerset, NJ (US); Sidney P. Foltis, Nutley, NJ (US)

Correspondence Address: Finnegan, Henderson, Farabow,, Garrett & Dunner, L.L.P., 1300 I Street, N.W., Washington, DC, 20005-3315, US

(21) Appl. No.: 10/424,111

(22) Filed: Apr. 28, 2003

Related U.S. Application Data
(60) Provisional application No. 60/375,416, filed on Apr. 26, 2002.

(51) Int. Cl. A61K 7/06; A61K 7/13

(52) U.S. Cl. 424/74; 8/406

(57) ABSTRACT

The present invention concerns cosmetic compositions and methods comprising an extract of Emblica officinalis and at least one ingredient chosen from dihydroxy acetone, a dibenzoyl methane derivative, ultrafine particles of zinc oxide, ultrafine particles of titanium oxide, astaxanthin, retinoids, alpha-hydroxy acids, beta-hydroxy acids, polyhydroxy acids, hydroquinone, compounds useful for the treatment of dandruff, hair colorants, hair pigments, and hair dyes. The addition of emblica extract to these compositions has the advantage of increasing their stability and therefore increasing or prolonging their effectiveness in such cosmetic compositions.

The present invention relates to cosmetic compositions comprising an extract of Emblica officinalis and to methods of using such compositions.

It is known that the exposure of skin and keratin fibers to ultraviolet-A (UV-A) radiation, with wavelengths from 320 nm and 400 nm, can tan the skin, but the exposure to UV-A radiation can also have an adverse effect on the skin, scalp, and keratin fibers over time. For example, exposure of the skin to UV-A radiation can cause a loss in the elasticity of the skin and the appearance of wrinkles, promoting a premature aging thereof. The UV-A rays may also cause the triggering of an erythemal reaction, or the reddening of skin in response to exposure to ultraviolet radiation, in certain individuals, and can also be the source of certain phototoxic or photoallergic reactions. Accordingly, it is desirable to prevent the exposure of skin to UV-A radiation.

A variety of organic sunscreen agents which absorb some UV-A irradiation are known. For example, derivatives of dibenzoylmethane, and, in particular, 4-(tert-butyl)-4′-methoxydibenzoyl methane, also known as avobenzone, are used in sunscreen compositions that can be applied to the skin. These compositions generally exhibit a high intrinsic absorption capacity for UV-A rays. It is known, however, that dibenzoyl methane derivatives can be unstable when exposed to ultraviolet radiation, especially UV-A, for long periods of time. For example, the dibenzoylmethane derivative avobenzone is known in the art as a useful ingredient in sunscreen compositions. But it is known that sunscreen compositions containing a mixture of avobenzone and other compounds useful as sunscreens, such as octyl methoxy cinnamate, are not always photostable. One solution to such a problem is to increase the concentration of the sun-screening agents in the sunscreen compositions. This solution, however, is not always feasible, as increasing the amount of such compounds can considerably increase the cost of such formulations and can also increase the chance of skin irritation.

This lack of photochemical stability of dibenzoyl methane derivatives to ultraviolet radiation necessarily limits their usefulness in sunscreen compositions, despite their advantage of exhibiting a high intrinsic absorption capacity. Accordingly, the use of these compounds in sunscreen compositions often requires that the user reapply the compositions at regular and frequent intervals to protect their skin from ultraviolet radiation.

The present invention overcomes these disadvantages of using dibenzoyl methane derivatives in sunscreen compositions by increasing their photostability through the addition of an extract of Emblica Officinalis, also known as Phyllanthus emblica, emblica, or the Indian gooseberry. The addition of emblica extract to the sunscreen compositions disclosed herein has the advantage of increasing the photostability of these compositions, thereby increasing or prolonging their effectiveness when they or their user are exposed to ultraviolet radiation.

Other sunscreen compositions comprise compounds that are known to be useful in reflecting or scattering ultraviolet rays, thereby protecting the skin, scalp, or keratin fibers from exposure to such rays. In particular, compositions comprising ultrafine particles of metal oxides, such as titanium dioxide and/or zinc oxide are known to be useful as sun-screening compositions. These compositions usually comprise ingredients other than the metal oxides in order to make them useful as cosmetic compositions. For example, such compositions may be in the form of a lotion or gel and therefore contain ingredients that are useful for suspending the ultrafine metal oxide particles. It is known in the art, however, that ultrafine particles of metal oxides can cause the oxidation of organic compounds in the presence of light, particularly ultra-violet light. Accordingly, one disadvantage of using ultrafine particles of metal oxides in cosmetic compositions comprising organic compounds is that the metal oxide particles can cause oxidation and degradation of the other cosmetic ingredients upon exposure to ultraviolet light. This is a particular problem in cosmetic compositions intended to function as sunscreens and containing organic compounds known to be useful as sun-screening agents. When such compositions also contain ultrafine metal oxide particles and are exposed to ultraviolet light, chemical degradation of the organic sun-screening agents can occur, thereby lessening the overall effectiveness of the composition in protecting the skin, scalp, or keratin fibers from the damaging effects of exposure to such light. According to the present invention, it has been found that the addition of an extract of Emblica officinalis to such compositions has the effect of decreasing the photocatalytic oxidation of other ingredients comprising the composition.

Furthermore, the present invention concerns the use of an extract Emblica officinalis in a cosmetic composition comprising hydroquinone. In general, hydroquinone compositions are known to be useful for the depigmenting of skin caused by, among other things, disorders of hyperpigmentation. One factor in determining skin color is the type and amount of melanin synthesized by the melanocyte and its distribution pattern in the surrounding keratinocytes. Melanin forms through a series of oxidative reactions involving the amino acid tyrosine in the presence of the enzyme tyrosinase. Tyrosinase converts tyrosine to dihydroxyphenylalanine (DOPA) and then to dopaquinone. Subsequently, dopaquinone is converted to dopachrome, through auto-oxidation, and finally to dihydroxyindole or dihydroxyindole-2-carboxylic acid (DHICA) to form eumelanin (brown-black pigment). This latter reaction occurs in the presence of dopachrome tautomerase and DHICA oxidase. In the presence of cysteine or glutathione, dopaquinone is converted to cysteinyl DOPA or glutathione DOPA, respectively.

Among skin-lightening or depigmenting agents, hydroquinone (HQ) is one of the most widely used. But, cosmetic compositions comprising hydroquinone are generally known to be unstable for long periods of time, especially at elevated temperatures. It has been found in the present invention that the addition of extract of Emblica officinalis has the advantage of increasing the stability of such hydroquinone-containing cosmetic compositions.

### SUMMARY OF THE INVENTION

The present invention concerns cosmetic compositions comprising an extract of Emblica officinalis and at least one ingredient chosen from dihydroxy acetone, a dibenzoyl methane derivative, ultrafine particles of zinc oxide, ultrafine particles of titanium oxide, astaxanthin, retinoids, alpha-hydroxy acids, beta-hydroxy acids, polyhydroxy acids, hydroquinone, compounds useful for the treatment of dandruff, hair colorants, hair pigments, and hair dyes. The addition of emblica extract to these compositions has the advantage of increasing their stability and therefore increasing or prolonging their effectiveness when they are used in such compositions.

In one embodiment, the present invention concerns cosmetic compositions comprising an extract of Emblica officinalis and dihydroxy acetone. In another embodiment, the present invention concerns cosmetic compositions comprising a dibenzoylmethane derivative and an extract of Emblica Officinalis. In another embodiment, the present invention concerns cosmetic compositions comprising an extract of Emblica officinalis and ultrafine particles of zinc oxide or ultrafine particles of titanium oxide. In yet another embodiment, the present invention concerns compositions comprising an extract of Emblica officinalis and astaxanthin. In still a further embodiment of the present invention, the at least one ingredient is chosen from retinoids. Further, in another embodiment, the present invention concerns a cosmetic composition comprising an extract of Emblica officinalis and at least one ingredient chosen from alpha-hydroxy acids, beta-hydroxy acids, and polyhydroxy acids. In a still further embodiment, the at least one ingredient is hydroquinone. In another embodiment, the present invention concerns cosmetic compositions comprising an extract of Emblica officinalis and compounds useful for the treatment of dandruff. Furthermore, the present invention concerns cosmetic compositions comprising an extract of Emblica officinalis and at least one ingredient chosen from hair colorants, hair pigments, and hair dyes.

The present invention also concerns methods of using cosmetic compositions comprising an extract of Emblica Officinalis. In one embodiment, the present invention concerns a method of stabilizing dihydroxy acetone, the methods comprising adding an extract of Emblica officinalis to the dihydroxy acetone.

Further, the present invention concerns methods for photostabilizing a cosmetic composition comprising a dibenzoylmethane derivative, comprising the addition of an extract of Emblica officinalis to such a composition.

Additionally, the present invention features methods for decreasing the photocatalytic effects of ultrafine particles of titanium dioxide and/or zinc oxide in cosmetic compositions containing them, comprising the addition of an extract of Emblica officinalis to such a composition.

The present invention also concerns a method of preventing the oxidation of a first composition, comprising adding to said first composition a second composition comprising an extract of Emblica officinalis and astaxanthin.

Furthermore, the present invention features methods of protecting the activity of and increasing the stability of compositions comprising retinoids or like compounds with vitamin A activity, the methods comprising adding an extract of Emblica officinalis to such a composition. Additionally, the present invention concerns methods of depigmenting skin comprising applying to the skin a composition comprising an extract of Emblica officinalis and retinoid compounds or like compounds with vitamin A activity.

Additionally, the present invention concerns methods of increasing the effectiveness and skin penetration of alpha-hydroxy acids, beta-hydroxy acids, polyhydroxy acids, comprising adding an extract of Emblica officinalis to compositions comprising such acids. Furthermore, the present invention concerns method of treating at least one skin condition chosen from acne, wrinkles, and signs associated with aging, said method comprising applying to the skin a composition comprising an extract of Emblica officinalis and at least one compound chosen from alpha-hydroxy acids, beta-hydroxy acids, and polyhydroxy acids.

The present invention also concerns methods for stabilizing compositions comprising hydroquinone, comprising adding an extract of Emblica officinalis to such compositions. Furthermore, the present invention concerns methods of depigmenting skin comprising applying to the skin a composition comprising an extract of Emblica officinalis and hydroquinone.

In addition, the present invention concerns methods of treating dandruff or dandruff-like conditions, comprising applying to the scalp a composition comprising an extract of Emblica officinalis and at least one compound useful for the treatment of dandruff or a dandruff-like condition.

The present invention also concerns methods of increasing the efficacy of hair colorants, hair pigments, and hair dyes, comprising applying such hair colorants, hair pigments, and hair dyes in the presence of an extract of Emblica officinalis.

Furthermore, the present invention concerns methods of protecting skin, scalp, or keratin fibers from damage due to exposure to ultraviolet radiation, comprising applying to the skin, scalp, or keratin fibers a composition comprising an extract of Emblica officinalis. The present invention especially concerns methods of protecting keratin fibers from ultraviolet radiation that were previously treated with hair colorants, hair pigments, and hair dyes, comprising applying to such keratin fibers, especially hair, an extract of Emblica officinalis.

### DETAILED DESCRIPTION OF THE INVENTION

As used herein, the terms “stabilizing” and “photostabilizing” mean to decrease the degradation of a particular ingredient contained in the compositions of the present invention by the addition of an extract of Emblica officinalis to compositions comprising the particular ingredient. The term “stability” can refer to the chemical stability and/or physical stability of such an ingredient in the presence and absence of such an extract. For example, particular ingredients in cosmetic compositions are known to undergo oxidation reactions over time in the presence of atmospheric oxygen. Such oxidation is undesirable because it both decreases the amount of such ingredient in the composition over time and can also form colored compounds, thereby giving the cosmetic composition an undesirable color. Additionally, some components of cosmetic compositions can undergo chemical degradation, such as oxidation, in the presence of light, such as ultraviolet light. Such degradation in the presence of light is undesirable since it has the effect of decreasing the amount of such an ingredient in the composition. This is a particular problem in cosmetic compositions formulated to act as sunscreening agents wherein exposure of the composition to ultraviolet light causes the degradation of ingredients meant to act as the sunscreening agents themselves.

The terms “photostabilization,” or “photostabilizing” mean to reduce the amount of chemical degradation with respect to a particular ingredient or ingredients contained in a cosmetic composition over time in the presence of an extract of Emblica officinalis, compared to the amount of degradation of the ingredient or ingredients in the absence of the extract when such a composition is exposed to light, particularly ultraviolet light.

As used herein, the terms “an extract of Emblica officinalis,” “emblica extract” or “an extract of emblica” mean any extract of the fruit of the Emblica officinalis or Phyllanthus emblica tree. Such extracts are described, for example, on pp. 175-176 of the Handbook of Ayurvedic Medicinal Plants, L. D. Kapoor, CRC Press, Inc., 1990. Additionally, such extracts are described in U.S. Pat. No. 6,261,605 B1 to Singh-Verma. Such extracts may be prepared by any method known to those skilled in the art. In addition, the terms “an extract of Emblica officinalis,” “emblica extract” and “an extract of emblica” mean any compounds or mixtures of compounds that are isolated or purified from crude extracts of the fruit of the tree. Such compounds or mixtures of compounds may be isolated or purified by any method known to those skilled in the art. For example, compositions comprising emblica extract, as well as its individual chemical components are described in U.S. Pat. No. 6,124,268, which is hereby incorporated by reference for this purpose.

As used herein, the term “dihydroxy acetone” means 1,3-dihydroxy-2-propanone. It is also specifically contemplated that this definition includes 1,3-dihydroxy-2-propanone in its dimeric form as well. Dihydroxy acetone is commercially available or can be prepared by any method known in the art.

As used herein, the term “dibenzoyl methane derivatives” means compounds that contain a dibenzoyl methane structure. These compounds are described, for example, in FR-A-2,326,405 and FR-A-2,440,933, and in EP-A-0,114,607. Such compounds are either commercially available or may be prepared by any method known to those skilled in the art. In addition, the term “dibenzoyl methane derivatives” means any mixture of two or more such compounds in any and all proportions.

In general, dibenzoyl methane derivatives that can be used according to the invention can be prepared by any method known to those skilled in the art. Such compounds are described, for example, in FR-2,326,405, FR-2,440,933 and EP-0,114,607, which are hereby incorporated by reference for that purpose. According to the present invention, such dibenzoyl methane derivatives may be chosen from, but are not limited to, at least one of 4-tert-butyl-4′-methoxy-dibenzoylmethane (also called 1-(p-tert-butylphenyl)-3-(p-methoxyphenyl)-1,3-propanedione, sold by Givaudan under the tradename “PARSOL 1789”), 4-isopropyldibenzoylmethane (sold by Merck under the tradename “EUSOLEX 8020”), 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyidibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoylmethane. In one embodiment, the at least one dibenzoyl methane derivative is 4-tert-butyl-4′-methoxy-dibenzoylmethane.

The compositions according to the present invention can contain an appropriate dibenzoyl methane derivative in an amount that ranges from about 0.01% to about 10% by weight relative to the total weight of the composition. Alternatively, such derivatives may be present in an amount ranging from about 0.1% to about 6% by weight relative to the total weight of the composition. The particular amount of a dibenzoyl methane derivative to be used according to the present invention will vary depending on the intended use and may vary depending on the other ingredients that comprise such a formulation. The selection of an appropriate dibenzoyl derivative and the amount thereof to be included according to the present invention are within the skill of those skilled in the art.

As used herein, the term “astaxanthin” means 3,3′-dihydroxy-β,β-carotene-4,4′-dione. This compound is commercially available (for example, from A.G. Scientific, Inc.) or may be prepared by any method known in the art.

The term “retinoids,” as used herein, means a class of compounds consisting of four isoprenoid units joined in a head-to-tail manner. In addition, it is specifically contemplated that the term “retinoids” also includes retinoids and retinoid-like compounds that exhibit qualitatively the biological activity of retinol, herein called “vitamin A activity.” Such compounds can be naturally occurring or may be synthetic.

As used herein, the term “alpha-hydroxy acids” means organic compounds that contain a hydroxy group in a position alpha to the carboxyl group. Such acids can be either naturally derived from various fruits, or they may be synthetic in nature. Examples of alpha-hydroxy acids that may be used according to the present invention include, but are not limited to glycolic acid, lactic acid, tartaric acid, citric acid, malic acid, and mandelic acid.

The term “beta-hydroxy acids,” as used herein, means organic acids that contain a hydroxy group in a position beta to the carboxyl group. Examples of beta-hydroxy acids that can be used according to the present invention include, but are not limited to, salicylic acid (or related derivatives such as salicylate, sodium salicylate, or willow extract), beta-hydroxybutanoic acid, tropic acid, and trethocanic acid.

As used herein, the term “poly-hydroxy acids” means organic acids that contain more than one hydroxyl ground and a carboxyl group. Poly-hydroxy acids that may be used according to the present invention include, but are not limited to, glyceric, dihydroxybutyric, ascorbic, glucuronic, mannuronic, tartronic, hydroxymalonic, malic, citramalic, hydroxyglutaric, tartaric, hydroxyfumaric, hydroxymaleic, dihydroxy maleic, dihydroxy fumaric, dihydroxy tartaric, citric and isocitric acids.

The term “hydroquinone,” as used herein, means 1,4-dihydroxybenzene. This compound is commercially available or can be prepared by any method known in the art.

As used herein, the term “depigmenting agent” means any compound that is useful in depigmenting the skin. Such agents include, but are not limited to, hydroquinone, kojic acid (5-hydroxy-4-pyran-4-one-2-methyl), azelaic acid, the monobenzyl ether of hydroquinone, N-acetyl-4-S-cysteaminylphenol, 4-hydroxyanisole, hydroquinone-beta-D-glucopyranoside, paper mulberry, glabridin (from licorice extract), extracts of Arctostaphylos patula and Arctostaphylos viscida, melatonin, and magnesium-L-ascorbyl-2-phosphate.

As used herein, the term “dandruff” means a chronic condition of the scalp characterized by excessive flaking, and sometimes also associated with itching and redness of the scalp. The term “compound useful in the treatment of dandruff” means compounds or compositions that are known to be useful in treating, preventing, or managing dandruff or a dandruff-like conditions. Such compounds may act by any mechanism known to have the effect of reducing flaking of the scalp, reducing scalp redness, or reducing itchiness of the scalp, or a combination of all three. Such compounds or compositions include, but are not limited to, antifungals (such as pyrithione zinc (ZPT), selenium sulfide, ketoconazole, climbazole, and octopirox), anti-proliferatives (such as coal tar), and keratolytic agents (such as sulfur and salicylic acid).

According to the present invention, compositions comprising an extract of emblica and at least one dibenzoyl methane derivative may also contain at least one other sun-screening agent. The term “sun-screening agent,” as used herein, means any compound or any combination of compounds which prevents or decreases the contact between UV-A and/or UV-B radiation and a surface, such as skin or keratin fibers, on which the compounds have been applied. Specifically included in this definition are compounds that prevent or reduce such contact by absorption and/or reflection and/or scattering of ultraviolet radiation, or any other mechanism known to those skilled in the art. These compounds may be organic or inorganic in nature. Additionally, the definition specifically includes mixtures of compounds that can prevent or reduce such contact by different mechanisms. For example, a composition according to the present invention can comprise a mixture of sun-screening agents, some of which are known to absorb ultraviolet radiation, while others are known to reflect or scatter such radiation.

Examples of at least one additional sun-screening agent that are believed to act by reflecting or scattering ultraviolet radiation include, but are not limited to, cerium oxides, chromium oxides, cobalt oxides, iron oxides, red petrolatum, silicone-treated titanium dioxide, titanium dioxide, zinc oxide, and/or zirconium oxide, or mixtures thereof. These compounds can be used in the present invention as discrete particles or as suspensions of such particles. In addition, the particle size and suspending agents employed will vary with the particular application in which they are used. The selection of the appropriate particle size and/or suspending agents is well within the skill in the art. A review of such compounds and their properties may be found at “Sun Protection Effect of Nonorganic Materials,” by S. Nakada & H. Konishi, Fragrance Journal, Volume 15, pages 64-70 (1987), which is incorporated by reference herein. In one embodiment of the present invention, the at least one additional sunscreening agent is zinc oxide in the form of ultrafine particles. In another embodiment, the at least one additional sunscreening agent is titanium oxide in the form of ultrafine particles.

The at least one other sunscreening agent that may be included in the compositions of the present invention and is believed to act by absorbing ultraviolet radiation includes, but is not limited to, those sunscreening agents that are known to absorb both UV-A or UV-B radiation. UV-A absorbers generally absorb radiation in the 320 to 400 nm region of the ultraviolet spectrum. Such UV-A absorbers include, but are not limited to, anthranilates, benzophenones, and dibenzoyl methane derivatives. UV-B absorbers generally absorb radiation in the 280 to 320 nm region of the ultraviolet spectrum. Such UV-B absorbers include, but are not limited to, p-aminobenzoic acid derivatives, camphor derivatives, cinnamates, and salicylates. It is specifically contemplated that the compositions and methods of the present invention may include mixtures of two or more compounds that are known to absorb ultraviolet radiation. For example, the compositions of the present invention may include a mixture of two or more compounds that are known to absorb UV-A radiation. Alternatively, the compositions of the present invention may include a mixture of compounds that are known to absorb UV-B radiation. Finally, the compositions of the present invention may include mixtures of two or more compounds, at least one of which is known to absorb UV-A radiation and at least one is known to absorb UV-B radiation.

The additional sunscreening agents may be chosen from, but are not limited to, cinoxate, diethanolamine p-methoxycinnamate, digalloyl trioleate, dioxybenzone, 2-ethoxyethyl-p-methoxycinnamate, ethyl 4-bis(hydroxypropyl)amino benzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid, 3-imidazol-4-ylacrylic acid ethyl ester, methyl anthranilate, octyldimethyl para-aminobenzoic acid, 2-phenylbenzimidazole-5-sulfonic acid and salts thereof, red petrolatum, sulisobenzone, titanium dioxide, triethanolamine salicylate, N, N, N-trimethyl-4-(2-oxoborn-3-ylidene methyl)anillinium methyl sulfate, para-aminobenzoic acid, oxyethylene-p-aminobenzoate, 2-ethylhexyl-p-dimethylaminobenzoate, ethyl N-oxypropylene-p-aminobenzoate, glycerol p-aminobenzoate, 4-isopropylbenzyl salicylate, 2-ethylhexyl 4-methoxycinnamate, methyl diisopropylcinnamate, isoamyl-4-methoxycinnamate, diethanolamine 4-methoxycinnamate, 3-(4′-trimethylammonium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, alpha.-(2-oxoborn-3-ylidene)-tolyl-4-sulfonic acid and soluble salts thereof, 3-(4′-sulfo)benzyliden-bornan-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl) anilino]-1,3,5-triazine, 2,4-bis{[4-(2-ethyl-hexyloxy)]-2-hydroxy]-phenyl}-6-(4-methoxy-phenyl)-1,3,5-triazine, the polymer of N-(2,4)-[(2-oxoborn-3-yliden)methyl]benzyl]-acrylamide, 1,4-bisbenzimidazolyl-phenylen-3,3′,5,5′-tetrasulfonic acid and salts thereof, benzalmalonate-substituted polyorganosiloxanes, benzotriazole-substituted polyorganosiloxanes, dispersed 2,2′-methylene-bis-[6-(2H-benzotriazol-2-yl)-4-(1,1,3,3-tetramethylbutyl)phenol], and solubilized 2,2′-methylene-bis-[6-(2H-benzotriazol-2-yl)-4-(methyl)phenol].

Examples of sun-screening compounds that are suitable for use in the present invention are described, for example, in U.S. Pat. Nos. 2,463,264, 4,367,390, 5,166,355 and 5,237,07, which are hereby incorporated by reference for that purpose. Additionally, such compounds are described in, for example, EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, also hereby incorporated by reference.

The other sun-screening agents that may be included according to the present invention may be present in an amount ranging from about 0.01% to about 30%% by weight relative to the total weight of the composition. Alternatively, such agents may be present in an amount ranging from about 0.1% to about 25% by weight relative to the total weight of the composition. Finally, such other agents may be present in amount ranging from 0.1% to 6% by weight relative to the total weight of the composition. The amount of a such a compound or compounds will vary depending on the intended use and may vary depending on the other ingredients that comprise such a formulation. The selection of an appropriate additional agent or agents and the amount thereof to be included according to the present invention are within the skill of those skilled in the art.

As used herein, the term “cosmetically acceptable salt” means salts that are not injurious to a user when applied to either the skin or scalp. Such salts may be prepared from organic or inorganic bases or organic or inorganic acids. Such salts are known to those skilled in the art and can be prepared by any known method. In addition, the term “cosmetically acceptable salt” means any mixture of such salts in any and all proportions unless otherwise stated.

In general, the compositions according to the present invention may be in the form of oil-in-water emulsions (namely, a cosmetically acceptable vehicle, carrier or diluent comprising an aqueous continuous dispersing phase and an oily discontinuous dispersed phase) which contain, in various concentrations, one or more standard lipophilic and/or hydrophilic organic sunscreen compounds capable of selectively absorbing harmful or deleterious UV radiation. The sunscreening compounds, and the amounts thereof, can be selected based on the desired sun protection factor (the sun protection factor (SPF) being expressed mathematically by the ratio of the irradiation time required to attain the erythema-forming threshold with the UV screening agent to the time required to attain the erythema-forming threshold in the absence of UV screening agent).

The cosmetic compositions according to the invention can take the form of oily or oleoalcoholic lotions, fatty or oleoalcoholic gels, solid sticks, emulsions such as a cream or a milk, vesicular dispersions of ionic or nonionic amphiphilic lipids, or an aerosol.

Such compositions may require the use of a solubilizing solvent. The solubilizing solvent may comprise an oil or a wax, a monohydric alcohol or a lower polyol or a mixture thereof may be used. Examples of monohydric alcohols or polyols that can be used according to the invention include, but are not limited to, ethanol, isopropanol, propylene glycol, and glycerol.

In another embodiment the composition is in the form of an oily lotion containing fatty acid esters, natural or synthetic oils and/or waxes, or oleoalcoholic lotions based on oils, on waxes or on fatty acid esters such as the triglycerides of fatty acids and of lower alcohols such as ethanol or of glycols such as propylene glycol and/or of polyols such as glycerol. The oleoalcoholic gels comprise an oil or a wax, an alcohol or a lower polyol, such as ethanol, propylene glycol or glycerol, and a thickener such as silica. The solid sticks are comprised of fats such as natural or synthetic waxes and oils, fatty alcohols, fatty acid esters and lanolin. The vesicular dispersions of ionic or nonionic amphiphilic lipids are prepared according to known processes, such as, for example, by causing the lipids to swell in an aqueous solution to form spherules dispersed in the aqueous medium, as described in the paper by BANGHAM, STANDISH and WATKINS, J. Mol. Biol., 13, 238 (1965) or in the French Patents FR 2,315,991 and 2,416,008.

In yet another embodiment, the compositions of the present invention are in the form of an emulsion or vesicular dispersion. In such forms the aqueous phase can contain water-soluble UV screening agents. Such agents include, but are not limited to, benzene-1,4-[di(3-methylidene-10-camphorsulfonic acid)], or cosmetically acceptable salts thereof, 2-phenylbenzimidazole-5-sulfonic acid, or cosmetically acceptable salts thereof, or 2-hydroxy-4-methoxybenzophenone-5-sulfonic acid, or cosmetically acceptable salts thereof.

In another embodiment, the composition is packaged as an aerosol. Such aerosols may use propellants that are known to those skilled in the art. Such propellants include, but are not limited to, alkanes, fluoroalkanes and chlorofluoroalkanes.

The compositions according to the invention can also contain metal oxide pigments which are dispersed throughout. The metal oxide pigments may be in the form of particles that can vary in size. For example, the metal oxide particles can be in the form of nanoparticles.

The topical cosmetic compositions of the present invention can comprise a carrier or mixture of carriers. The carrier should be cosmetically and/or pharmaceutically acceptable, meaning that the carrier is suitable for topical application onto the skin, scalp, or keratin fibers, has good aesthetic properties, is compatible with the other ingredients of the present invention, and will not cause any unwanted safety or toxicity concerns. The carriers and additional components used to formulate compositions according to the present invention will vary with the product type and its intended use. Such choices are within the skill of one skilled in the art. The carriers can comprise from about 0.5% to about 99.5% by weight, or from about 5.0% to about 99.5% by weight, or from about 10.0% to about 98.0% by weight, relative to the total weight of the composition. As used herein, the phrase “suitable for topical application onto skin, the scalp or keratin fibers,” means that the carrier does not damage or negatively affect the aesthetics of or cause irritation to skin, the scalp, or keratin fibers.

Carriers suitable for use with the present invention include, but are not limited to, those known in the art as being useful in the formulation of a wide variety of product types, including creams, dispersions, emulsions, gels, lotions, milks, mousses, sprays, and tonics.

The carriers used herein can comprise components used in conventional cosmetic and/or dermatological compositions. For example, they can comprise a solvent to dissolve or disperse the chosen ingredients. They may also comprise additional materials including, but not limited to, esters (such as isopropyl myristate), halogenated hydrocarbons (such as freons), hydrocarbons (such as decene, hexane, and isobutane), linalool, and volatile silicon derivatives such as phenyl pentamethyl disiloxane, methoxypropyl heptamethyl cyclotetrasiloxane, chloropropyl pentamethyl disiloxane, hydroxypropyl pentamethyl disiloxane, octamethyl cyclotetrasiloxane, decamethyl cyclopentasiloxane, cyclomethicone, dimethicone, and mixtures thereof.

Mousses and aerosol sprays according to the present invention can include any propellants known to those skilled in the art to be useful for the delivery of the composition as a foam, in the case of a mousse, or as a fine, uniform spray, in the case of an aerosol spray. Examples of suitable propellants include, but are not limited to, hydrofluorinated compounds, dichlorodifluoromethane, difluoroethane, dimethylether, isobutane, n-butane, propane, or trichlorofluromethane. A tonic or spray product having a low viscosity may also include an emulsifying agent. Examples of suitable emulsifying agents include, but are not limited to, anionic surfactants, cationic surfactants, nonionic surfactants, and mixtures thereof. If the composition is a formulated such that it contains a relatively low level of volatile organic solvents, such as alcohols, and relatively high levels of water (i.e., in excess of about 10%, by weight, water), fluorosurfactants may be advantageously used. If such a fluorosurfactant is used, it can be present in an amount from about 0.01% to about 7.5% by weight relative to the total weight of the composition. The amount of a suitable propellant can be adjusted depending on the particular composition ingredients and its intended use. Such choices are well within the skill of one skilled in the art, but the propellant is generally present in an amount from about 3% to about 30% by weight of mousse compositions and from about 15% to about 50% by weight relative to the total weight of the aerosol spray compositions.

The compositions according to the present invention can include conventional, non-aerosol pump sprays, i.e., “atomizers,” aerosol containers or cans having propellant, and pump aerosol containers utilizing compressed air as the propellant. Pump aerosol containers are disclosed, for example, in U.S. Pat. Nos. 4,077,441 and 4,850,517, both of which are herein incorporated by reference for this purpose.

The compositions according to the present invention can also comprise a therapeutically effective amount of a pharmaceutical additive or adjuvant. The term “therapeutically effective,” as used herein, means a sufficient amount of an active agent to treat, prevent, or manage a particular medical condition, while avoiding unwanted side effects. Generally, a therapeutically effective amount of a pharmaceutically active agent will vary according to the specific agent being used, the ability of the agent to penetrate through a subject's skin or scalp, the age and physical condition of the subject being treated, the condition being treated, prevented, or managed, the severity of the condition, the duration of the treatment, the nature of concurrent therapy, and other factors known to those skilled in the art. The determination of a therapeutically effective amount of such a component is within the skill of one skilled in the art.

The compositions according to the present invention can also be in the form of an emulsion and can comprise at least one compound useful as an emulsifier. Suitable emulsifiers that can be used according to the present invention include, but are not limited to, nonionic, cationic, anionic, and zwitterionic emulsifiers. Suitable emulsifiers according to the present invention include, but are not limited to, acyl lactylates, alkyl phosphates, carboxylic acid copolymers, esters and ethers of glucose, esters of glycerin, esters of propylene glycol, esters of sorbitan anhydrides, esters of sorbitol, ethoxylated ethers, ethoxylated alcohols, fatty acid amides, fatty acid esters of polyethylene glycol, fatty esters of polypropylene glycol, polyoxyethylene fatty ether phosphates, soaps and mixtures thereof. Other emulsifiers that may be used include, but are not limited to, PPG-2 isoceteth-20 acetate, ceteareth-20, ceteth-10, cetyl phosphate, diethanolamine cetyl phosphate, glyceryl stearate, PEG-100 stearate, polyethylene glycol 20 sorbitan monolaurate, polyethylene glycol 5 soya sterol, polysorbate 60, polysorbate 80, potassium cetyl phosphate, PPG-2 methyl glucose ether distearate, steareth-20, and mixtures thereof. In one embodiment, the emulsifier is PPG-2 isoceteth-20 acetate. For examples of other suitable emulsifiers that can be used according to the present invention, see, for example, McCutcheon's, Detergents and Emulsifiers, North American Edition (1986), Allured Publishing Corporation; and U.S. Pat. Nos. 5,011,681, 4,421,769, and 3,755,560.

The compositions according to the invention can also comprise at least one emollient. Suitable emollients include, but are not limited to, branched hydrocarbons, non-polar carboxylic acid and alcohol esters, volatile and non-volatile silicone oils, and mixtures thereof. See, for example, U.S. Pat. No. 4,919,934, which is incorporated by reference for this purpose. Examples of emollients that can be used according to the present invention include, but are not limited to, at least one of octyldodecyl neopentanoate and propylene glycol isoceteth-3 acetate.

The compositions according to the present invention can also comprise emollients, organic solvents, demulcents, antioxidants, opacifying agents, stabilizers, alpha-hydroxy acids, moisturizers, vitamins, fillers, insect repellents, silicones, cationic polymers and thickeners, softeners, anti-foaming agents, hydrating agents, chelators, gums and thickeners, low pH thickening agents, polymers for enhancing the film-forming properties and substantivity of the composition, sequestrants, skin penetrating aids, suspending agents, vitamins and derivatives thereof, preservatives, humectants, surfactants, oils, fats, waxes, lanolin, fragrances, propellants, anionic, cationic, nonionic or amphoteric polymers or mixtures thereof, basifying or acidifying agents, dyes, metal oxide pigments of particle size between 100 nm and 20,000 nm such as iron oxides, or other colorings and/or pigments whose function is to color the composition itself, color the skin, or color keratin fibers, such as the hair, or any other ingredient known to those skilled in the art that are used in cosmetic compositions. These ingredients are selected so that they do not interfere with or exert any substantial adverse effect on the light stabilization elicited by the emblica extracts contained in the compositions of the present invention.

The fats that may be used according to the present invention may consist of an oil or a wax or a mixture thereof, fatty acids, fatty acid esters, fatty alcohols, petrolatum, paraffin, lanolin, hydrogenated lanolin or acetylated lanolin. The oils may be chosen from, but are not limited to, animal, plant, mineral and synthetic oils. For example, the oils include, but are not limited to, hydrogenated palm oil, hydrogenated castor oil, liquid petrolatum, paraffin oil, purcellin oil, volatile and non-volatile silicone oils, poly-alpha-olefins, fluorinated oils, perfluorinated oils, and isoparaffins. The waxes can be chosen from, but are not limited to, animal, fossil, plant, mineral, and synthetic waxes. For example, the waxes include, but are not limited to, bees waxes, carnauba wax, candelila wax, sugar cane wax, Japan wax, ozokerites, montan wax, microcrystalline waxes, paraffins, and silicone resins and waxes.

Preservatives that may be used according to the present invention are those which are known to those skilled in the art useful for the prevention or retardation of microbial growth. Such preservatives can prevent spoilage of the present compositions. For examples of such suitable preservatives, see, for example, CFTA International Cosmetic Ingredient Dictionary and Handbook, seventh edition, 2, 1654 (1997).

The optional organic solvents can include, but are not limited to alcohols and polyols. In one embodiment, the organic solvent is a lower polyol. The thickeners include, but are not limited to crosslinked polyacrylic acids, guar gums and celluloses, modified or otherwise, such as hydroxypropylated guar gum, methylhydroxyethylcellulose, hydroxypropylmethylcellulose and hydroxyethylcellulose.

The compositions according to the present invention can be applied to the skin, scalp, or keratin fibers, such as hair, in a conventional fashion to provide the desired benefit. Such methods of use can utilize compositions that are in a form suitable for topical administration including, but not limited to, a lotion, cream, gel, or spray. For example, an effective amount of a composition according to the present invention can be sprayed or applied to the skin. The composition is allowed to remain on the skin until it is absorbed or it can be removed at later time. Alternatively, such compositions can be applied to the scalp or keratin fibers and allowed to remain thereon until it is absorbed or it can be removed at a later time.

The compositions according to the present invention can be used for protecting the skin, the scalp, or keratin fibers, such as hair, against ultraviolet radiation. As such it can be specifically formulated as a sunscreen product or as a make-up product or both.

When the compositions according to the present invention are applied to keratin fibers, such as the hair, they can be in the form of a shampoo, a lotion, a gel, an emulsion, a vesicle dispersion or a lacquer for the hair and may comprise, for example, a composition to be rinsed, to be applied before or after shampooing, before or after dyeing or bleaching, or before, during or after permanent-waving or straightening of the hair, a styling or treating lotion or gel, a lotion or gel for blow drying or hairsetting, or a composition for the permanent-waving, straightening, dyeing or bleaching of the hair.

When the compositions according to the invention are used as a make-up product for the eyelashes, the eyebrows or the skin, such as a treatment cream for the epidermis, a foundation, a lipstick, an eyeshadow, a blusher, a mascara or an eyeliner, they may be in solid or pasty, anhydrous or aqueous form, such as oil-in-water or water-in-oil emulsions, vesicle dispersions, or suspensions.

The present invention also concerns a method of protecting skin, the scalp, or keratin fibers against ultraviolet radiation. The method comprises the applying a composition according to the present invention, which comprises an extract of Emblica officinalis and at least one other ingredient chosen from dihydroxy acetone, a dibenzoyl methane derivative, ultrafine particles of zinc oxide, ultrafine particles of titanium oxide, astaxanthin, retinoids, alpha-hydroxy acids, beta-hydroxy acids, polyhydroxy acids, hydroquinone, compounds useful for the treatment of dandruff, hair colorants, hair pigments, and hair dyes, to the skin, the scalp or to keratin fibers. The compositions and methods according to the present invention have the advantage of protecting the skin, scalp, or keratin fibers from damage due to exposure to ultraviolet radiation for a longer time period than is possible in such compositions not containing an emblica extract. All of the at least one additional ingredients that may be used in the methods according to the present invention are described above. In one embodiment, the at least one other ingredient is 4-(tert-butyl)-4′-methoxydibenzoyl methane.

The present invention also concerns a method for photostabilizing a composition comprising a dibenzoyl methane derivative, comprising adding an extract of Emblica officinalis to said composition. In one embodiment, the dibenzoyl methane derivative is 4-(tert-butyl)-4′-methoxydibenzoyl methane.

The present invention also concerns a method of stabilizing dihydroxy acetone, comprising adding an extract of Emblica officinalis to said dihydroxy acetone.

The present invention also concerns a method of reducing the photocatalytic effects of at least one ingredient chosen from ultrafine particles of zinc oxide and ultrafine particles of titanium oxide in a cosmetic composition, said method comprising adding an extract of Emblica officinalis to said composition.

The present invention further concerns a method of preventing the oxidation of a first composition, comprising adding to said first composition a second composition comprising an extract of Emblica officinalis and astaxanthin.

The present invention also concerns a method of stabilizing compositions comprising retinoids and retinoid-like compounds, comprising adding an extract of Emblica officinalis. The addition of an extract of Emblica officinalis to such compositions has the advantage of protecting their activity, acting in conjunction such compounds as effective depigmenting agents, and increasing the chemical stability of such compositions.

The present invention also concerns a method of increasing the skin penetration of a composition comprising alpha-hydroxy acids, beta-hydroxy acids, and poly-hydroxy acids, said method comprising adding an extract of Emblica officinalis to such a composition.

Additionally, the present invention concerns a method of treating at least one skin condition chosen from acne, wrinkles, and signs associated with aging, comprising applying to the skin a composition comprising an extract of Emblica officinalis and at least one additional ingredient chosen from alpha-hydroxy acids, beta-hydroxy acids, and poly-hydroxy acids. It is specifically contemplated that the cosmetic compositions according to the present invention can comprise an alpha-hydroxy acid, in combination with a beta-hydroxy acid, and/or a poly-hydroxy acid. In addition, it is specifically contemplated that the compositions according the present invention can comprise more than one alpha-hydroxy acid, in combination more than one beta-hydroxy acid, and/or more than one poly-hydroxy acid. The number and identity of each type of acid will depend on the desired properties of such compositions as well as their intended use. The choice of a particular acid or acids is within the skill of one skilled in the art.

The present invention also concerns a method of treating dandruff, comprising applying to the scalp a composition comprising an extract of Emblica officinalis and at least one compound or composition useful for the treatment of dandruff. The compounds or compositions useful for treating dandruff that can be used according to the present invention include, but are not limited to, compounds that act by any mechanism known to have the effect of reducing flaking of the scalp, reducing scalp redness, or reducing itchiness of the scalp, or a combination of all three. Such compounds or compositions include, but are not limited to, antifungals (such as pyrithione zinc (ZPT), selenium sulfide, ketoconazole, climbazole, and octopirox), anti-proliferatives (such as coal tar), and keratolyic agents (such as sulfur and salicylic acid). It is specifically contemplated that the compositions of the present invention may comprise one or more compounds that are useful in the treatment of dandruff, including two or more compounds that are known to act via different mechanisms in treating, preventing, or managing dandruff or dandruff-like conditions. For example, compositions according to the present invention may comprise at least one compound known to be useful as an antifungal agent, and at least one compound known to be useful as an anti-proliferative agent, or at least one compound known to be useful as a keratolyic agent, or a combination of at least one compound from all three classes. For example, a composition according to the present invention may comprise pyrithione zinc, coal tar, sulfur and an extract of Emblica officinalis. It is also specifically contemplated that the compositions according to the present invention can comprise more than one compound useful in the treatment of dandruff that act by the same mechanism. For example, a composition according to the present invention may comprise two or more compounds known to act as antifungals, such as pyrithione zinc and selenium sulfide. The choice of the specific compounds to be used will depend on factors known to those skilled in the art, such as the condition being treated, its severity, and the age and condition and the subject being treated.

The present invention also concerns a method of coloring keratin fibers, comprising applying to the keratin fibers a composition comprising an extract of Emblica officinalis and at least one dye, colorant, or pigment.

Furthermore, the present invention concerns a method of protecting color-treated keratin fibers, comprising applying to the color-treated keratin fibers a composition comprising an extract of Emblica officinalis. It is contemplated that such an extract may be applied to the keratin fibers after they have been color-treated, or it may be applied before the fibers have been so treated. Additionally, the extract of Emblica officinalis may be applied in the form of a lotion, cream, gel, or conditioner, which is allowed to remain on the keratin fibers throughout the time during which they will be exposed to ultraviolet radiation. Alternatively, the extract of Emblica officinalis may be applied in the form of a cream, lotion, gel, or shampoo that is subsequently rinsed out of the hair prior to exposure to ultraviolet radiation. In the latter case, it is contemplated that a sufficient amount of emblica extract remains on the keratin fibers after rinsing that the hair is protected from exposure to ultraviolet radiation.

The present invention further concerns a method for depigmenting skin, comprising applying to the skin a composition comprising an extract of Emblica officinalis and at least one compound known to be useful for depigmenting skin. The at least one compound known to be useful for depigmenting skin can be chosen from, but is not limited to, hydroquinone, kojic acid (5-hydroxy-4-pyran-4-one-2-methyl), azelaic acid, the monobenzyl ether of hydroquinone, N-acetyl-4-S-cysteaminylphenol, 4-hydroxyanisole, hydroquinone-beta-D-glucopyranoside, paper mulberry, glabridin (from licorice extract), extracts of Arctostaphylos patula and Arctostaphylos viscida, melatonin, and magnesium-L-ascorbyl-2-phosphate. In one embodiment, the depigmenting agent is hydroquinone. It is contemplated that the compositions according the present invention can comprise at least one additional depigmenting agent in addition to hydroquinone. The particular choice of additional depigmenting agent or agents to include in the composition comprising hydroquinone and an extract of Emblica officinalis will depend on such factors as the desired chemical stability of such a composition, the potential toxicological effects of such compositions, and the overall depigmenting effectiveness of such compositions. Such information is readily available to those skilled in the art or can be determined by those skilled in the art without undue experimentation.

The present invention also concerns a method for increasing the chemical stability of a cosmetic composition comprising hydroquinone, comprising adding an extract of Emblica officinalis to the composition. In addition, it is specifically contemplated that the extract of Emblica officinalis can be used to increase the chemical stability of a cosmetic composition comprising hydroquinone and at least one additional depigmenting agent. The at least one additional depigmenting agent can be chosen from, but is not limited to, kojic acid (5-hydroxy-4-pyran-4-one-2-methyl), azelaic acid, the monobenzyl ether of hydroquinone, N-acetyl-4-S-cysteaminylphenol, 4-hydroxyanisole, hydroquinone-beta-D-glucopyranoside, paper mulberry, glabridin (from licorice extract), extracts of Arctostaphylos patula and Arctostaphylos viscida, melatonin, and magnesium-L-ascorbyl-2-phosphate, comprising the addition of an extract of Emblica officinalis to the composition.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

### Example 1

Increased Photostability of 4-tert-butyl-4′-methoxy-dibenzoylmethane in the Presence of an Extract of Emblica officinalis

A total of three cosmetic compositions were prepared. Composition A contained no extract of Emblica officinalis, composition B contained 0.5 w/w % of extract of Emblica officinalis, and composition C contained 1 w/w % of extract of Emblica officinalis. The ingredients contained in each composition are shown below.

| | Composition A | Composition B | Composition C |
|---|---|---|---|
| Ingredients | (w/w %) | (w/w %) | (w/w %) |
| Deionized water | 75 | 74.58 | 74.1 |
| Disodium EDTA | 0.1 | 0.1 | 0.1 |
| Methyl paraben | 0.3 | 0.3 | 0.3 |
| Chlorphenesin | 0.2 | 0.2 | 0.2 |
| Phenoxy ethanol | 0.7 | 0.7 | 0.7 |
| Propylene glycol | 4.0 | 4.0 | 4.0 |
| Glycerin | 4.0 | 4.0 | 4.0 |
| Xanthan Gum | 0.07 | 0.07 | 0.07 |
| Butyl | 3.0 | 3.0 | 3.0 |
| methoxy- | | | |
| dibenzoylmethane | | | |
| (avobenzone) | | | |
| Octyl salicylate | 5.0 | 5.0 | 5.0 |
| Propyl paraben | 0.1 | 0.1 | 0.1 |
| Glyceryl sterate and | 1.0 | 1.0 | 1.0 |
| PEG-100 | | | |
| DC 200 (dimethicone) | 0.5 | 0.5 | 0.5 |
| Stearic acid | 1.0 | 1.0 | 1.0 |
| Ganex 220 | 1.3 | 1.3 | 1.3 |
| Cyclopentasiloxane | 2.0 | 2.0 | 2.0 |
| Pemulen TR-1 | 0.2 | 0.2 | 0.2 |
| Deionized water | 1.0 | 1.0 | 1.0 |
| Triethanolamine | 0.45 | 0.45 | 0.45 |
| Extract of Emblica | 0.0 | 0.5 | 1.0 |
| Officinalis | | | |

Samples of each composition were placed in an Atlas Heraeus Suntest CPS instrument and exposed to ultraviolet radiation for a total of 2 h. Samples (0.15 g) of each composition were removed from the instrument at 30 min, 60 min, 90 min, and 120 min. The samples were dissolved in 100 mL of isopropanol and the resulting mixture was sonicated until a homogenous solution was obtained. A portion of the solution (4 mL) was placed into a 50 mL volumetric flask and the resulting solution was sonicated for 5 min. The absorbance of the solution was measured in a Perkin-Elmer spectrophotometer. The resulting absorbance values for each solution at each time point is shown below.

| | Composition A | Composition B | Composition C |
|---|---|---|---|
| Initial | 1.163 | 1.091 | 1.060 |
| absorbance | | | |
| (t = 0) | | | |
| Absorbance | 0.418 (64.06%) | 0.481 (55.95%) | 0.488 (53.93%) |
| at 30 min | | | |
| (% loss) | | | |
| Absorbance | 0.158 (86.39%) | 0.178 (83.71%) | 0.195 (81.62%) |
| at 60 min | | | |
| (% loss) | | | |
| Absorbance | 0.116 (90.0%) | 0.131 (87.95%) | 0.125 (88.22%) |
| at 90 min | | | |
| (% loss) | | | |
| Absorbance | 0.112 (90.36%) | 0.118 (89.18%) | 0.103 (90.29%) |
| at 120 min | | | |
| (% loss) | | | |

Compositions B and C, containing 0.5 w/w % and 1 w/w % extract of Emblica officinalis, respectively, showed a significant improvement in the photostability of the avobenzone-containing compositions at the 30 minute time point.

### Example 2

Stabilizing Effect of Emblica officinalis Extract on Compositions Containing Hydroquinone

Three compositions were prepared that contained 2 w/w % of hydroquinone, one with no extract of Emblica officinalis and one with 0.5 w/w % of Emblica officinalis extract. The compositions were then exposed to atmospheric conditions at 25° C., 37° C., and 50° C. The compositions were then observed for a color change, darkening for instance, indicating degradation.

| | Composition A | Composition B | Composition C |
|---|---|---|---|
| Ingredient | (w/w %) | (w/w %) | (w/w %) |
| Water | QS | QS | QS |
| Potato starch-modified | 2.0 | 2.0 | 2.0 |
| Dicaprylyl ether | 0.5 | 0.5 | 0.5 |
| C₁₂-C₁₅ alkyl | 4.0 | 4.0 | 4.0 |
| benzoate | | | |
| Octyl palmitate | 3.0 | 3.0 | 3.0 |
| Cetyl dimethicone | 1.0 | 1.0 | 1.0 |
| Cetyl phosphate | 2.0 | 2.0 | 2.0 |
| (Amphisol A) | | | |
| Glycerol | 1.5 | 1.5 | 1.5 |
| monostearate-SE | | | |
| Cetyl alcohol | 1.0 | 1.0 | 1.0 |
| Stearic acid | 1.0 | 1.0 | 1.0 |
| Preservatives | 0.8 | 0.8 | 0.8 |
| Triethyl amine | 0.4 | 0.4 | 0.4 |
| Hydroquinone | 2.0 | 2.0 | 2.0 |
| Emblica | 0.0 | 0.25 | 0.5 |

After 2 days at 25° C., 37° C., and 50° C., the composition containing no emblica extract showed a color change at all temperatures, indicating degradation. The samples containing 0.25 w/w % emblica extract showed some color change at all temperatures, but it was not as visible in comparison to the samples containing no emblica extract. The samples containing 0.5 w/w % emblica extract showed no color change at any temperature.

### Example 3

Reduction in the Photocatalytic Effects of Titanium Dioxide in the Presence of Emblica Extract

Two compositions were prepared, both containing 5 w/w % of titanium dioxide, one containing no emblica extract and the other containing 0.5 w/w % of emblica extract. An 11 g sample of each composition was removed and were placed into an Atlas Heraeus Suntest CPS instrument and exposed to ultraviolet radiation for a total of 6 h. Samples of each composition were then tested for color change using a Minolta CM-2600d instrument. The composition of each sample is shown in the table.

| | Composition A | Composition B |
|---|---|---|
| Ingredient | (w/w %) | (w/w %) |
| PEG-60 lanolin | 7.0 | 7.0 |
| C₁₂-C₁₅ alkyl benzonate | 7.0 | 7.0 |
| Glyceryl stearate and PEG- | 4.0 | 4.0 |
| 100 | | |
| Stearyl alcohol | 1.0 | 1.0 |
| Cyclopentasiloxane | 3.0 | 3.0 |
| Deionized water | QS | QS |
| Preservatives | 1.0 | 1.0 |
| Xanthan Gum | 0.2 | 0.2 |
| Glycerin | 5.0 | 5.0 |
| Emblica extract | 0.5 | — |
| ultrafine titanium dioxide | 5.0 | 5.0 |
| (coated with aluminum | | |
| hydroxide and stearic acid) | | |

The composition that did not contain emblica extract, Composition B, showed considerable darkening and became more green and less yellow in color. This sample showed a significant ΔE*ab of 6.83, which upon inspection was visible to the naked eye. In contrast, Composition A, containing 0.5 w/w % of emblica extract also became darker and less red. It showed a ΔE*ab of 0.74. The color change, however, was not visible to the naked eye. The addition of emblica extract to these compositions significantly decreased the photocatalytic activity of the titanium dioxide contained therein and as a result the composition suffered less degradation.

We claim:

1. A cosmetic composition comprising an extract of Emblica officinalis and at least one ingredient chosen from dihydroxy acetone, dibenzoyl methane derivatives, ultrafine particles of zinc oxide, ultrafine particles of titanium oxide, astaxanthin, retinoids, hydroquinone, alpha-hydroxy acids, beta-hydroxy acids, polyhydroxy acids, compounds useful for the treatment of dandruff, hair colorants, hair pigments, and hair dyes.

2. A cosmetic composition according to claim 1, wherein the at least one dibenzoyl methane derivative is chosen from 4-tert-butyl-4′-methoxy-dibenzoylmethane, 4-(1,1-dimethylethyl)-4′-methoxydibenzoylmethane, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyidibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoylmethane.

3. A cosmetic composition according to claim 2, wherein the at least one dibenzoyl methane derivative is chosen from 4-tert-butyl-4′-methoxy-dibenzoylmethane.

4. A cosmetic composition according to claim 1, wherein the at least one ingredient is dihydroxy acetone.

5. A cosmetic composition according to claim 1, wherein the at least one ingredient is chosen from ultrafine particles of zinc oxide and ultrafine particles of titanium oxide.

6. A cosmetic composition according to claim 1, wherein the at least one ingredient is astaxanthin.

7. A cosmetic composition according to claim 1, wherein the at least one ingredient is a retinoid compound.

8. A cosmetic composition according to claim 1, wherein the at least one ingredient is chosen from alpha-hydroxy acids, beta-hydroxy acids, and polyhydroxy acids.

9. A cosmetic composition according to claim 1, wherein the at least one ingredient is hydroquinone.

10. A cosmetic composition according to claim 9, further comprising at least one additional skin depigmenting agent.

11. A cosmetic composition according to claim 1, wherein the at least one ingredient is a compound useful for the treatment of dandruff.

12. A cosmetic composition according to claim 1, wherein the at least one ingredient is chosen from hair colorants, hair pigments, and hair dyes.

13. A cosmetic composition according to claim 1, comprising at least one additional sunscreen compound.

14. A cosmetic composition according to claim 13, wherein said at least one additional sunscreen compound absorbs light with a wavelength of 320 nm to 400 nm or 280 nm to 320 nm.

15. A cosmetic composition according to claim 13, comprising a first additional sunscreen compound that absorbs light with a wavelength of 320 nm to 400 nm and a second additional sunscreen compound that absorbs light with a wavelength of 280 nm to 320 nm.

16. A cosmetic composition according to claim 13, wherein said at least one additional sunscreen compound is chosen from cinoxate, diethanolamine p-methoxycinnamate, digalloyl trioleate, dioxybenzone, 2-ethoxyethyl p-methoxycin namate, ethyl 4-bis(hydroxypropyl)aminobenzoate, 2-ethylhexyl-2-cyano-3,3-diphenylacrylate, ethylhexyl p-methoxycinnamate, 2-ethylhexyl salicylate, glyceryl aminobenzoate, homomenthyl salicylate, homosalate, 3-imidazol-4-ylacrylic acid, 3-imidazol-4-ylacrylic acid ethyl ester, methyl anthranilate, octyldimethyl p-aminobenzoic acid, 2-phenylbenzimidazole-5-sulfonic acid and salts thereof, red petrolatum, sulisobenzone, titanium dioxide, triethanolamine salicylate, N, N, N-trimethyl-4-(2-oxoborn-3-ylidene methyl)anillinium methyl sulfate, para-aminobenzoic acid, oxyethylene-p-aminobenzoate, 2-ethylhexyl p-dimethylaminobenzoate, ethyl N-oxypropylene p-aminobenzoate, glycerol p-aminobenzoate, 4-isopropylbenzyl salicylate, 2-ethylhexyl 4-methoxycinnamate, methyl diisopropylcinnamate, isoamyl 4-methoxycinnamate, diethanolamine 4-methoxycinnamate, 3-(4′-trimethylammonium)-benzyliden-bornan-2-one methylsulfate, 2-hydroxy-4-methoxybenzophenone, 2-hydroxy-4-methoxybenzophenone-5-sulfonate, 2,4-dihydroxybenzophenone, 2,2′,4,4′-tetrahydroxybenzophenone, 2,2′-dihydroxy-4,4′dimethoxybenzophenone, 2-hydroxy-4-n-octoxybenzophenone, 2-hydroxy-4-methoxy-4′-methoxybenzophenone, alpha.-(2-oxoborn-3-ylidene)-tolyl-4-sulfonic acid and soluble salts thereof, 3-(4′-sulfo)benzyliden-bornan-2-one and soluble salts thereof, 3-(4′-methylbenzylidene)-d,1-camphor, 3-benzylidene-d,1-camphor, benzene 1,4-di(3-methylidene-10-camphosulfonic) acid and salts thereof, urocanic acid, 2,4,6-tris[p-(2′-ethylhexyl-1′-oxycarbonyl)-anilino]-1,3,5-triazine, 2-[(p-(tertiobutylamido)anilino]-4,6-bis-[(p-(2′-ethylhexyl-1′-oxycarbonyl)anilino]-1,3,5-triazine, 2 ,4-bis{[4-(2-ethyl-hexyloxy)]-2-hydroxy]-phenyl)-6-(4-methoxy-phenyl )-1,3,5-triazine, the polymer of N-(2 et 4)-[(2-oxoborn-3-yliden)methyl]benzyl]-acrylamide, 1,4-bisbenzimidazolyl-phenylen-3,3′,5,5′-tetrasulfonic acid and salts thereof, benzalmalonate-substituted polyorganosiloxanes, benzotriazole-substituted polyorganosiloxanes, dispersed 2,2′-methylene-bis-[6-(2H-benzotriazol-2-yl)-4-(1,1,3,3-tetramethylbutyl)phenol], and solubilized 2,2′-methylene-bis-[6-(2H-benzotriazol-2-yl)-4-(methyl)phenol].

17. A method of protecting skin, scalp, or keratin fibers against ultraviolet radiation, comprising applying to skin, scalp, or keratin fibers a composition comprising an extract of Emblica officinalis and at least one ingredient chosen from a sunscreening agent.

18. A method according to claim 17, wherein the at least one sunscreening agent is at least one dibenzoylmethane derivative.

19. A method according to claim 18, wherein said at least one dibenzoyl methane derivative is chosen from 4-tert-butyl-4′-methoxy-dibenzoylmethane, methylethyl)-4′-methoxydibenzoyl 4-(41,13-dimmethane 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyidibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoylmethane.

20. A method according to claim 19, wherein the at least one dibenzoylmethane derivative is 4-tert-butyl-4′-methoxy-dibenzoylmethane.

21. A process for photostabilizing a composition comprising at least one dibenzoyl methane derivative, said process comprising adding an extract of Emblica officinalis to said composition.

22. A method according to claim 21, wherein said at least one dibenzoyl methane derivative is chosen from 4-tert-butyl-4′-methoxy-dibenzoylmethane, 4-(1,1 -dimethylethyl)-4′-methoxydibenzoylmethane, 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-dimethoxydibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane, and 2,6-dimethyl-4-tert-butyl-4′-methoxydibenzoyl methane.

23. A method according to claim 22, wherein said at least one dibenzoyl methane derivative is 4-tert-butyl-4′-methoxy-dibenzoylmethane.

24. A method of stabilizing dihydroxy acetone, comprising adding an extract of Emblica officinalis to said dihydroxy acetone.

25. A method of reducing the photocatalytic effects of ultrafine particles of zinc oxide or ultrafine particles of titanium dioxide, comprising adding an extract of Emblica officinalis to a composition comprising said ultrafine particles of zinc oxide or ultrafine particles of titanium dioxide.

26. A method of preventing the oxidation of a first composition, comprising adding to said first composition a second composition comprising an extract of Emblica officinalis and astaxanthin.

27. A method of stabilizing compositions comprising hydroquinone, comprising adding an extract of Emblica officinalis to said compositions.

28. A method of depigmenting skin, comprising applying to the skin a composition comprising an extract of Emblica officinalis and at least one depigmenting compound.

29. A method according to claim 29, wherein said at least one depigmenting compound is chosen from hydroquinone, 5-hydroxy-4-pyran-4-one-2-methyl, azelaic acid, the monobenzyl ether of hydroquinone, N-acetyl-4-S-cysteaminylphenol, 4-hydroxyanisole, hydroquinone-beta-D-glucopyranoside, paper mulberry, glabridin, extracts of Arctostaphylos patula and Arctostaphylos viscida, melatonin, magnesium-L-ascorbyl-2-phosphate, and retinoids.

30. A method according to claim 29, wherein said at least one depigmenting agent is hydroquinone.

31. A method of protecting the activity of and increasing the stability of compositions comprising retinoids, comprising adding an extract of Emblica officinalis to said composition.

32. A method of increasing the skin penetration of alpha-hydroxy acids, beta-hydroxy acids, or poly-hydroxy acids, comprising adding an extract of Emblica officinalis to compositions comprising such acids.

33. A method of treating at least one skin condition chosen from acne, wrinkles, and signs associated with aging, comprising applying to the skin a composition comprising an extract of Emblica officinalis and at least one compound chosen from alpha-hydroxy acids, beta-hydroxy acids, and poly-hydroxy acids.

34. A method of treating dandruff, comprising applying to the scalp a composition comprising an extract of Emblica officinalis and at least one compound useful for the treatment of dandruff.

35. A method according to claim 34, wherein said at least one compound useful for the treatment of dandruff is chosen from antifungals, anti-proliferatives, and keratolyic agents.

36. A method according to claim 35, wherein said antifungals are chosen from pyrithione zinc, selenium sulfide, ketoconazole, climbazole, and octopirox.

37. A method according to claim 35, wherein said anti-proliferatives are chosen from coal tar.

38. A method according to claim 35, wherein said keratolyic agents are chosen from sulfur and salicylic acid.

39. A method of coloring keratin fibers, comprising applying to the keratin fibers a composition comprising an extract of Emblica officinalis and at least one dye, colorant, or pigment.

40. A method of protecting color-treated keratin fibers, comprising applying to the color-treated keratin fibers a composition comprising an extract of Emblica officinalis.
